(19) United States
(12) Patent Application Publication
(10) Pub. No.: US 2020/0016344 A1
(43) Pub. Date: Jan. 16, 2020

(54) PORTABLE DEVICE FOR INHALATION OF AT LEAST ONE ACTIVE COMPOSITION

(71) Applicant: ENOVAP, Rosieres Pres Troyes (FR)

(72) Inventors: ALEXANDRE SCHECK, Rosieres Pres Troyes (FR); Julien ABULFEDA, Rosieres Pres Troyes (FR); VALENTIN DELAHAYE, Rosieres Pres Troyes (FR); JEAN-BAPTISE MATTE, Rosieres Pres Troyes (FR); ALEXIS FISCHER, Rosieres Pres Troyes (FR); AYMARD DE LACROIX DE RAVIGNAN, Rosieres Pres Troyes (FR); MARIE HARANG, Rosieres Pres Troyes (FR); DAMIEN MONTANARI, Rosieres Pres Troyes (FR)

(21) Appl. No.: 16/498,240

(22) PCT Filed: Apr. 3, 2018
(86) PCT No.: PCT/FR2018/050828

§ 371 Date: Sep. 26, 2019

(30) Foreign Application Priority Data
Mar. 31, 2017 (FR) 1752797
Mar. 31, 2017 (FR) 1752799

(51) Int. Cl. A61M 15/00; A61M 11/04

(52) U.S. Cl. CPC A61M 15/0003; A61M 2205/33; A61M 11/041; A61M 15/009

(57) ABSTRACT

The portable device (100, 200, 300, 400) for inhalation of at least one active composition comprises:
at least one reservoir (105, 110, 205) for storing an active composition,
an intermediate chamber (115) connected to at least one reservoir,
an inhalation line (120) connected to the intermediate chamber,
a means (130, 135, 230) for aerosolising the active composition coming from a reservoir, and
a regulator (125, 225, 325, 405, 410) for regulating the quantity of aerosolised active composition according to a command representing a quantity of active composition to be aerosolised received by the regulator.

### TECHNICAL FIELD OF THE INVENTION

The present invention relates to a portable device for inhalation of at least one active composition. It applies, in particular, to the field of medical devices.

### STATE OF THE ART

Inhalation is a technique for administering therapeutic treatments that has been utilized for thousands of years.

Inhalation enables active substances to be delivered quickly and efficiently to the circulatory system via the lungs. The main advantage of this technique is the speed of action of the active substance inhaled, compared to oral administration for example. Unlike substances administered orally, the inhaled substances do not pass through the liver, which destroys many molecules of interest.

Inhalation is also the most effective avenue of treatment for administering active substances locally for treating pulmonary illnesses.

The drawback of current inhalers is that they do not allow the dosage of the dose administered to the patient to be adjustable.

Among the therapeutic inhalers available in the market, a majority are based on techniques of aerosolization by depressurization of a compressed gas containing the active substance or by nebulization of the active substance. The pressurized inhalers and nebulizers have a significant advantage over personal vaporizers, because the aerosolized active substances are not subjected to thermal degradation. Better control of the aerosolization process can be of relevance for certain therapies using active substances sensitive to thermal degradation.

### SUBJECT OF THE INVENTION

The present invention aims to remedy all or part of these drawbacks.

To this end, the present invention envisages a portable device for inhalation of at least one active composition, which comprises:
at least one reservoir for storing an active composition;
at least one intermediate chamber connected to at least one reservoir;
an inhalation line connected to the intermediate chamber;
a means for aerosolizing the active composition coming from a reservoir; and
a regulator for regulating the quantity of aerosolized active composition according to a command representing a quantity of active composition to be aerosolized received by the regulator.

Thanks to these provisions, the quantity of active composition inhaled can be directly regulated inside the portable device. In particular, the utilization of several reservoirs and, for each reservoir, a means for transforming the active composition into an aerosol that can be inhaled, makes it possible to combine the advantages of each type of aerosolization in a single device. When a single active composition is used and there are two reservoirs, it is possible to dose the quantity of active substance as a function of the supply to the intermediate chamber from at least one reservoir.

In some embodiments, at least one aerosolization means is a nebulizer for liquid active composition stored in a reservoir.

These embodiments enable an aerosol to be formed without thermal degradation of the active composition.

In some embodiments, the nebulizer comprises a screen having openings, and a means for making the screen oscillate, the regulator comprising a means for closing at least one portion of the openings according to the quantity of active composition to be aerosolized.

These embodiments make it possible to control the quantity of active composition nebulized.

In some embodiments, the nebulizer comprises a means for making a surface oscillate, the regulator being configured to make the surface oscillate at a frequency determined as a function of the quantity of active composition to be aerosolized and/or of characteristics of the active composition to be nebulized.

These embodiments make it possible to control the quantity of active composition nebulized.

In some embodiments, the surface is a screen having openings, the regulator being configured to make the screen oscillate at a frequency determined as a function of the quantity and/or characteristics of the active composition to be nebulized.

These embodiments make it possible to control the quantity of active composition nebulized.

In some embodiments, the surface is located in the bottom portion of the reservoir so as to transmit vibrations to the active composition, the regulator being configured to command the oscillation at a frequency determined as a function of the quantity and/or characteristics of the active composition to be nebulized.

These embodiments make it possible to control the quantity of active composition nebulized.

In some embodiments, the nebulizer comprises a plurality of screens having different porosities, and a means for making each screen oscillate independently, the regulator being configured to command the oscillation of at least one of the screens as a function of the quantity and/or characteristics of the active composition to be nebulized.

In some embodiments, the device that is the subject of the present invention comprises a vaporizer for liquid active composition stored in a reservoir.

These embodiments make it possible to combine both a nebulized active composition and a vaporized active composition.

In some embodiments, the device that is the subject of the present invention comprises at least two reservoirs holding an active composition to be aerosolized, one holding an active composition containing an active substance and the other holding an active composition containing a lower, or zero, concentration of said active substance, each aerosolization means associated with a said reservoir being controlled as a function of the quantity and/or characteristics of the active composition to be aerosolized.

In some embodiments, the device that is the subject of the present invention comprises a pressurized reservoir, holding oxygen or any other pressurized gas that can contain an active substance in the form of a solution or suspension, connected to the intermediate chamber, the aerosolization of the oxygen or pressurized gas being controlled by the regulator.

These embodiments enable the user to inhale the oxygen or any other pressurized gas in addition to the active composition.

In some embodiments, at least two reservoirs are pressurized, one holding a pressurized active composition containing an active substance and the other holding a pressurized active composition containing a lower, or zero, concentration of said active substance, at least one reservoir being connected to the intermediate chamber acting as expansion chamber for the fluids being released there.

In some embodiments, the regulator is configured to command a duration for aerosolizing the active composition coming from at least one of the two reservoirs as a function of a quantity of active substance, contained in the composition, to be delivered.

These embodiments make it possible to control the quantity of active composition inhaled by the user.

In some embodiments, the device that is the subject of the present invention comprises at least two reservoirs, each reservoir containing a different active composition.

These embodiments make it possible to combine, in one inhalation, the effects of two active compositions.

In some embodiments, the device that is the subject of the present invention comprises at least two aerosolization means.

In some embodiments, at least two aerosolization means are each connected to a separate reservoir, the device comprising at least two reservoirs.

In some embodiments, at least a first aerosolization means is a nebulizer and at least a second aerosolization means is a nebulizer.

In some embodiments, at least a first aerosolization means is a means for expanding a pressurized content and at least a second aerosolization means is a means for expanding a pressurized content.

In some embodiments, at least two aerosolization means utilize a different aerosolization technique.

In some embodiments, at least a first aerosolization means is a nebulizer and at least a second aerosolization means is a vaporizer.

In some embodiments, at least a first aerosolization means is a nebulizer and at least a second aerosolization means is a means for expanding a pressurized content in a reservoir.

In some embodiments, at least a first aerosolization means is a vaporizer and at least a second aerosolization means is a means for expanding a pressurized content in a reservoir.

In some embodiments, at least one vaporizer is a cup vaporizer heated by conduction or convection.

In some embodiments, at least one vaporizer is a heating element vaporizer.

In some embodiments, at least one vaporizer is a heating mesh vaporizer.

In some embodiments, the intermediate chamber stores a heated compound following the activation of at least one vaporizer for active composition contained in a reservoir.

In some embodiments, the intermediate chamber comprises an element heating the compound stored in said chamber.

In some embodiments, the heating element is positioned near a mesh or a cup containing the compound.

In some embodiments, the heating element is positioned at least in part around and/or in the center of the compound.

In some embodiments, the intermediate chamber can be removed from the device.

### BRIEF DESCRIPTION OF THE FIGURES

Other advantages, aims and particular features of the invention will become apparent from the non-limiting description that follows of at least one particular embodiment of the device that is the subject of the present invention, with reference to drawings included in an appendix, wherein:

FIG. 1 represents, schematically, a first particular embodiment of the device that is the subject of the present invention;

FIG. 2 represents, schematically, a second particular embodiment of the device that is the subject of the present invention;

FIG. 3 represents, schematically, a third particular embodiment of the device that is the subject of the present invention;

FIG. 4 represents, schematically, a fourth particular embodiment of the device that is the subject of the present invention;

FIG. 5 represents, schematically, a fifth particular embodiment of the device that is the subject of the present invention;

FIG. 6 represents, schematically, a sixth particular embodiment of the device that is the subject of the present invention;

FIG. 7 represents, schematically, a seventh particular embodiment of the device that is the subject of the present invention;

FIG. 8 represents, schematically, an eighth particular embodiment of the device that is the subject of the present invention; and

FIG. 9 represents, schematically, a ninth particular embodiment of the device that is the subject of the present invention.

### DESCRIPTION OF EXAMPLES OF REALIZATION OF THE INVENTION

The present description is given in a non-limiting way, each characteristic of an embodiment being able to be combined with any other characteristic of any other embodiment in an advantageous way.

It is now noted that the figures are not to scale.

“Active composition” means here any composition comprising at least one active substance active in solid, liquid or gaseous form, or any combination of these states, said substance comprising an active molecule whose administration, by inhalation to a patient, has a therapeutic effect. Such an active substance is, for example:
nicotine;
an opioid; or
THC (for “Δ-9-tetrahydrocannabinol”).

When this active substance has a liquid form, it can be diluted in a base containing, for example, saline water, N-acetylcysteine and/or an isotonic solution, PG (for “Propylene Glycol”) or VG (for “Vegetable Glycerin”). Aromatic agents can be added to this base. Similarly, water-stable vitamins or aqueous plant extracts can be added to this active substance. This assembly thus forms the active composition. The active composition can have a liquid, solid or gaseous form. For example, the active composition can have a concentrated oil form.

When this active substance is contained in a compressed gas in liquid form, it can be in solution (solubilized in the compressed gas) or in suspension (particles of active substance are suspended in the compressed liquid gas). An aromatic agent can be added to this base, as well as vitamins stable in the gas.

The active composition can also take the form of extracts of plants and/or dried plants.

Although not numbered, each embodiment of the device, 100, 200, 300 and 400, 500, 600, 700 and 800, comprises a battery providing the autonomous electrical power supply for all the electrical and electronic components of the device.

As can be understood from reading the following description, the device that is the subject of the present invention comprises at least one reservoir, provided with a means for aerosolizing the active composition contained in said reservoir. Preferably, the device comprises at least two reservoirs, each reservoir having either a different active composition active, or a different aerosolization means. This makes it possible to combine the advantages of two different aerosolization means or two different active compositions in a single inhalation and by means of a single device.

Aerosolization here means, for example, the nebulization of a composition, the vaporization of a composition, or the depressurization of a pressurized composition in a reservoir.

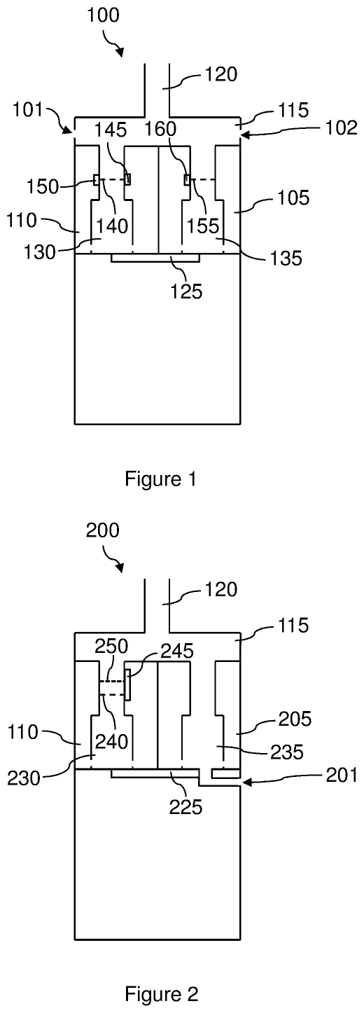

FIG. 1, which is not to scale, shows a schematic view of an embodiment of the device 100 that is the subject of the present invention. This portable device 100 for inhalation of at least one active composition comprises:
at least one reservoir, 105 and/or 110, for storing an active composition;
at least one intermediate chamber 115 connected to at least one reservoir;
an inhalation line 120 connected to at least one intermediate chamber;
a means, 130 and/or 135, for aerosolizing the active composition coming from a reservoir; and
a regulator 125 for regulating the quantity of aerosolized active composition according to a command representing a quantity of active composition to be aerosolized received by the regulator.

Each reservoir, 105 and/or 110, is, for example, a sealed volume provided with an opening connected to the intermediate chamber 115. In some variants, each reservoir, 105 and/or 110, also comprises an inlet for supplying an active composition in which the active substance can be more or less diluted.

When the device 100 comprises a nebulizer of the active composition contained in a reservoir, 105 or 110, the opening of this reservoir, 105 or 110, is connected to a nebulizer, the nebulizer being connected to the intermediate chamber 115.

The device 100 shown in FIG. 1 comprises two reservoirs, 105 and 110. However, the device 100 can comprise a single reservoir, 105 or 110, or more than two reservoirs, at least one reservoir being connected to the intermediate chamber.

The intermediate chamber 115 is, for example, a sealed volume having at least two types of openings: one corresponding to openings connecting the intermediate chamber 115 to at least one reservoir, 105 and/or 110, and the other corresponding to the opening connecting the intermediate chamber 115 to the inhalation line 120.

When the device 100 comprises a nebulizer of the active composition in liquid form contained in a reservoir, 105 and/or 110, the intermediate chamber 115 preferably comprises at least one opening, 101 and/or 102, acting as air inlet during the patient's suction on the inhalation line 120. These openings, 101 and 102, can be replaced by at least one opening at the location of at least one reservoir, when such a reservoir is provided, for example, with an active composition vaporizer. These air inlets, 101 and/or 102, make it possible to cause the nebulized active composition coming from a reservoir, 105 and/or 110, to flow towards the inhalation line 120. The vibration of at least one membrane, 140 or 155, can be controlled during reception of a command by the regulator 125. This command can be issued during the pressing of a button, on the device 100, by the user or by a third-party terminal, such as a smartphone for example.

The regulator 125 is, for example, an electronic control circuit configured to, as a function of the command received, trigger the aerosolization of a defined quantity of active composition from each reservoir, 105 and/or 110, in the intermediate chamber 115. The nature of the command issued by the regulator 125 depends on the state of the active composition stored in each reservoir, 105 and/or 110, and on any transformation of this active composition carried out in the device 100.

The command received by the regulator 125 can be received via an antenna configured to receive electromagnetic signals emitted using the IEEE 802.11 standard, known as “Wi-Fi”, or Bluetooth (registered trademark) standard, for example. Alternatively, the antenna is configured to receive any wireless signal emitted over a point-to-point connection or via an internet type of data network or a mobile data network.

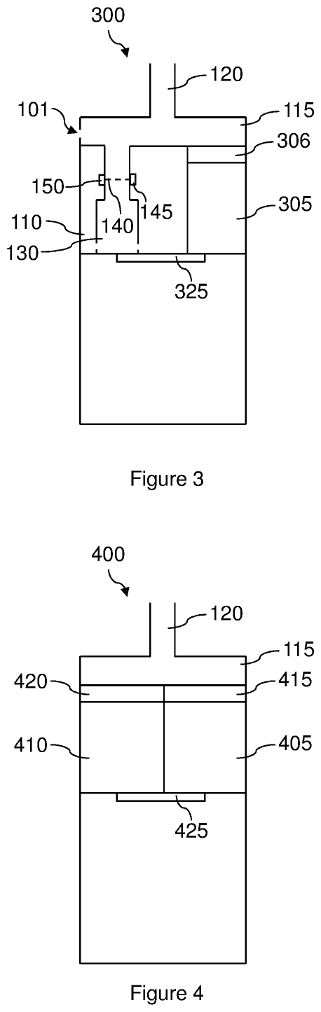

In a variant, as shown in FIG. 3, the device 300 comprises a single reservoir 305 of an active composition having the form of a gas once released, the regulator 125 opening the passage between the reservoir 305 and the intermediate chamber for a duration determined as a function of the quantity of active composition to be aerosolized.

In another variant, as shown in FIG. 4, the device 400 comprises two reservoirs, 405 and 410, each comprising an active composition containing different concentrations of the same active composition. Depending on the quantity of active substance to be aerosolized, the regulator 125 enables the aerosolization of a determined volume of active composition coming from each reservoir, 105 and 110, such that, at a constant volume inhaled by the user, the quantity of active substance aerosolized differs according to the aerosolization from each reservoir.

In the embodiment shown in FIG. 1, at least one aerosolization means is a nebulizer 130 for the active composition stored in the reservoir 110. The device 100, as shown in FIG. 1, also comprises a nebulizer 135 for the active composition contained in the reservoir 105. In some variants, the device 100 comprises only one of these nebulizers, 130 or 135.

It is recalled here that a nebulizer is a device making it possible to transform certain liquids into a cloud of extremely fine particles, or haze of particles, when cold. For ultrasonic nebulizers, the transformation is carried out by means of a vibrating surface actuated by ultrasonics. For pneumatic nebulizers, the nebulization is generated by introducing a gas into the active composition to be nebulized.

In some variants, the nebulizer, 130 and 135, comprising at least one surface, membrane or screen, 140 and/or 155, is made to oscillate under the constraint of an oscillation means, 145 or 160, such as a piezoelectric crystal for example. In these variants, the surface, membrane or screen is preferably located above the active composition.

The regulator 125 is then configured to command the oscillation of the surface at a frequency determined as a function of the quantity of active composition to be aerosolized.

In the case of this screen nebulizer, the vibration causing the oscillation of the membrane perforated with openings or of the screen having openings results, by capillarity through the membrane or screen, in the formation of droplets of active composition in suspension in the intermediate chamber 115.

The regulator 125 is then configured to command the oscillation of the screen at a frequency determined as a function of the quantity and/or characteristics of the active composition to be nebulized.

In some variants, the surface is located in the bottom portion of the reservoir so as to transmit vibrations to the active composition, the regulator 125 being configured to command the oscillation at a frequency determined as a function of the quantity and/or characteristics of the active composition to be nebulized.

In these variants, at least one nebulizer, 130 and/or 135, comprises for example a non-porous cup containing a volume of active composition to be nebulized connected to a reservoir, 105 and/or 110. The vibration means, 145 and/or 160, which can consist of a piezoelectric metal that can be located in the bottom portion of the reservoir, causes the formation of jets on the surface of the active composition, these jets resulting in the formation of droplets staying above the surface of the active composition. The vibration means, 145 and/or 160, which can be located in the bottom portion of the reservoir, transmits the vibrations to the active composition to be nebulized, either directly or by means of a fluid coupling.

In this latter case, the active composition is deposited in a hemispheric cup partially immersed in a water reservoir placed above the crystal: the water acts as transmission liquid.

When a nebulizer is employed, the active composition takes the form of a liquid composition. This composition can be liquid initially or result from the melting of an initially solid composition, for example.

When the device 100 comprises at least two nebulizers, 130 and 135, each associated reservoir, 105 and 110, stores a composition having a different concentration of active substance. In this way, the quantity of active substance inhaled by the patient is regulated by nebulizing a different volume of liquid coming from each reservoir, 105 and 110, such that at a constant total nebulized volume, the quantity of active substance varies depending on the proportion of the volume coming from each reservoir, 105 and 110.

In a first example, the composition of the first reservoir comprises an 80% concentration of an active substance and the second reservoir comprises a 20% concentration of the active substance. In order to administer a volume of nebulized solution having a 50% concentration, the regulator 125 commands the nebulization of an identical volume coming from each reservoir. To obtain a volume of nebulized solution having a 60% concentration, the regulator 125 commands the nebulization of a volume coming from the first reservoir equal to twice the volume coming from the second reservoir.

In a second example, the composition of the first reservoir comprises an 80% concentration of an active substance and the second reservoir comprises a 0% concentration of the active substance. In order to administer a volume of nebulized solution having a 40% concentration, the regulator 125 commands the nebulization of an identical volume coming from each reservoir. To obtain a volume of nebulized solution having a 60% concentration, the regulator 125 commands the nebulization of a volume coming from the first reservoir equal to three times the volume coming from the second reservoir.

In some embodiments, such as that shown in FIG. 1, a nebulizer 130 comprises a screen 140 having openings, and a means 145 for making the screen oscillate, the regulator 125 comprising a means 150 for closing at least one portion of the openings according to the quantity of active composition to be aerosolized.

This closing is achieved, for example, by closing valves forming all or part of the screen. These valves, sensitive to an electrical current passing over it, are opened or closed according to the value of the current's intensity or voltage.

This closing makes it possible to limit the flowrate of the droplet haze formed and therefore the quantity of nebulized active composition inhaled by the patient relative to the aspirated air coming from the air inlet 101 and/or to the quantity of nebulized active composition from at least one other reservoir 105 if there is another such reservoir 105.

In some variants, each nebulizer, 130 and 135, operates in an identical manner to the nebulizer 130 as described above.

In some embodiments, such as that shown in FIG. 1, a nebulizer 135 comprises a screen 155 having openings, and a means 160 for making the screen oscillate, the regulator 125 being configured to make the screen oscillate at a frequency determined as a function of the quantity and/or characteristics of the active composition to be aerosolized.

The quantity and/or characteristics of the nebulized active composition depends on the oscillation frequency of the screen 155. In this way, this frequency variation makes it possible to adjust the flowrate and/or characteristics of the droplet haze formed and therefore the quantity and/or characteristics of the nebulized active composition inhaled by the patient relative to the aspirated air coming from the air inlet 101 and/or to the quantity and/or characteristics of the nebulized active composition from at least one other reservoir 110 if there is another such reservoir 110.

In some embodiments, such as that shown in FIG. 1, a nebulizer 135 comprises an oscillation means 160, the regulator 125 being configured to command oscillation at a frequency determined as a function of the quantity and/or characteristics of the active composition to be aerosolized.

The quantity and/or characteristics of the nebulized active composition depends on the oscillation frequency. In this way, this frequency variation makes it possible to adjust the flowrate and/or characteristics of the droplet haze formed and therefore the quantity and/or characteristics of the nebulized active composition inhaled by the patient relative to the aspirated air coming from the air inlet 101 and/or to the quantity and/or characteristics of the nebulized active composition from at least one other reservoir 110 if there is another such reservoir 110.

In some variants, each nebulizer, 130 and 135, operates in an identical manner to the nebulizer 135 as described above.

In this way, as can be understood, regulating the quantity of active composition determined can be carried out:
when the device 100 comprises several reservoirs, by regulating the ratio of flowrates of nebulized active composition by each reservoir; and
when the device 100 comprises several reservoirs, by regulating the ratio of flowrates of nebulized active composition in one reservoir relative to a flowrate of another aerosolization of the active composition, as described with reference to FIG. 2 or FIG. 3 for example.

FIG. 2, which is not to scale, shows a schematic view of an embodiment of the device 200 that is the subject of the present invention. This portable device 200 for inhalation of at least one active composition comprises:
at least one reservoir, 205 and/or 110, for storing an active composition;
at least one intermediate chamber 115 connected to at least one reservoir;
an inhalation line 120 connected to at least one intermediate chamber;
a means, 240 and/or 235, for aerosolizing the active composition coming from a reservoir; and
a regulator 225 for regulating the quantity of aerosolized active composition according to a command representing a quantity of active composition to be aerosolized received by the regulator.

The reservoir 110, intermediate chamber 115 and inhalation line 120 are identical to the reservoir 110, intermediate chamber 115 and inhalation line as described with reference to FIG. 1.

This embodiment comprises a nebulizer 230 comprising a plurality of membranes, or screens, 240 and 250, provided with openings allowing the passage of the active composition contained in the reservoir 110. These screens, 240 and 250, can be arranged in parallel along a line connecting the reservoir 110 to the intermediate chamber 115. Each screen, 240 and 250, has a different porosity. This porosity can depend on the number of openings in each screen, 240 and 250. Alternatively, for each screen, 240 and 250, the dimensions of the openings of these screens, 240 and 250, differ in length, height and/or width. During the oscillation of one or other of these membranes, this difference in dimension causes the nebulization of the active composition according to a flowrate and/or characteristics of the aerosol that varies as a function of the porosity of said screen. In this way, the characteristics of the nebulized haze can be controlled as well as the ratio of this flowrate to the flowrate of air aspirated by an inlet (not shown) in the intermediate chamber 115 and/or to the nebulization flowrate of another nebulizer, 130 and/or 135, or another means for aerosolizing the active composition.

The device 200 also comprises a means 245 for making each screen oscillate independently, the regulator 225 being configured to command the oscillation of at least one of the screens as a function of the quantity and/or characteristics of the active composition to be aerosolized. This oscillation means 245 operates in an identical manner to the oscillation means 145 as described with reference to FIG. 1. If each screen is used independently, the haze droplets do not necessarily pass through the two screens. One of the screens can be moved so as not to come into contact with the haze produced by the other screen.

This device 200 optionally comprises a second reservoir 205 provided with a vaporizer 235 of the active composition contained in said reservoir 205. This second reservoir 205 comprises an air inlet 201 replacing the air inlet, as described above, in the intermediate chamber 115.

The vaporizer 235 is, for example, a heater resistor configured to heat the active composition and, according to the heating duration and temperature, cause a determined vaporization flowrate and/or characteristics. This heating duration and/or temperature are controlled by the regulator 225. The active composition to be heated is initially liquid.

In some variants, the vaporizer 235 is, for example, a bowl heated by conduction or convection. In these variants, the active composition initially comes, for example, from plants or in an oily concentrate or as a wax. Such an oil or wax represents, for example, a concentrate coming from plants.

In some variants, the vaporizer 235 is, for example, a mesh heated by conduction or convection. In these variants, the active composition is, for example, initially oily, such as essential oils or THC oil, for example.

The regulator 225 determines and commands, for example, a ratio of the flowrates or quantity of nebulization/vaporization of the liquid contained in each reservoir, 110 and 205. This flowrate, or this quantity, is possibly subordinated to a captured flowrate of inhalation by the patient or to a command received by the regulator 225.

Structurally, the regulator 225 is, for example, identical to the regulator 125.

FIG. 3, which is not to scale, shows a schematic view of an embodiment of the device 300 that is the subject of the present invention. This portable device 300 for inhalation of at least one active composition comprises:
at least one reservoir, 305 and/or 110, for storing an active composition;
at least one intermediate chamber 115 connected to at least one reservoir;
an inhalation line 120 connected to at least one intermediate chamber;
a means, 130 and/or 135, for aerosolizing the active composition coming from a reservoir; and
a regulator 325 for regulating the quantity of aerosolized active composition according to a command representing a quantity of active composition to be aerosolized received by the regulator.

The reservoir 110, intermediate chamber 115 and inhalation line 120 are identical to the reservoir 110, intermediate chamber 115 and inhalation line as described with reference to FIG. 1.

The device 300 also comprises a nebulizer 130 that is similar to the nebulizer 130 as described with reference to FIG. 1. In some variants, this nebulizer 130 is replaced by the nebulizer 135 as described with reference to FIG. 1 or the nebulizer 230 as described with reference to FIG. 2.

This device 300 also comprises a reservoir 305 of pressurized oxygen, connected to the intermediate chamber 115, the aerosolization of oxygen being commanded by the regulator 325. This oxygen reservoir 305 can be replaced by any other reservoir of pressurized gas able to contain an active composition, or not.

The aerosolization of oxygen can be carried out by a piston or by opening a valve.

The regulator 325 determines and commands, for example, a ratio of the flowrate or quantity of nebulization of the liquid contained in the reservoir 110, or of the flowrate or quantity of pressurized gas aerosol coming from the reservoir 305. This flowrate, or this quantity, is possibly subordinated to a captured flowrate of inhalation by the patient or to a command received by the regulator 325.

Structurally, the regulator 325 is, for example, identical to the regulator 125.

FIG. 4, which is not to scale, shows a schematic view of an embodiment of the device 400 that is the subject of the present invention. This portable device 400 for inhalation of at least one active composition comprises:
at least one reservoir, 405 and/or 410, for storing an active composition;
at least one intermediate chamber 115 connected to at least one reservoir;
an inhalation line 120 connected to at least one intermediate chamber;
a means, 415 and/or 420, for aerosolizing the active composition coming from a reservoir; and
a regulator 425 for regulating the quantity of aerosolized active composition according to a command representing a quantity of active composition to be aerosolized received by the regulator.

The intermediate chamber 115 and inhalation line 120 are identical to the intermediate chamber 115 and inhalation line as described with reference to FIG. 1.

In this device 400, at least two reservoirs 405, 410 are pressurized, one holding a pressurized active composition containing an active substance and the other holding a pressurized active composition containing a lower, or zero, concentration of said active substance, at least one reservoir being connected to at least one intermediate chamber 115 acting as expansion chamber for the fluids entering therein.

The regulator 425 determines and commands, for example, a ratio of the flowrates or durations of aerosolization of the fluid contained in the reservoirs 405 and 410. The regulator 425 is configured to command a flowrate and/or duration of aerosolization from at least one of the two reservoirs, 405 and 410, as a function of the quantity of active composition to be aerosolized.

Structurally, the regulator 425 is, for example, identical to the regulator 125.

In some variants, the device, 100, 200, 300 and/or 400, comprises at least two reservoirs, 110, 105, 205, 305, 405 and 410, each reservoir containing a different active composition.

For example, in a first reservoir there is a primary active composition comprising a primary active substance to be regulated and a secondary active substance, and in a second reservoir there is an active composition that only comprises the secondary active substance, which allows a precise concentration of the primary active substance to be administered and the secondary active substance to be administered continuously.

In this way, as can be seen by reading the description given above, the quantity of the aerosolized active composition can be controlled via a communicating terminal, such as a smartphone or personal computer, for example. This spontaneous control can be incorporated into a program aimed at regulating the quantity of active composition inhaled over time. In these variants, prior to each inhalation, the communicating terminal transmits to the regulator of the device the quantity to be released for inhalation by the patient.

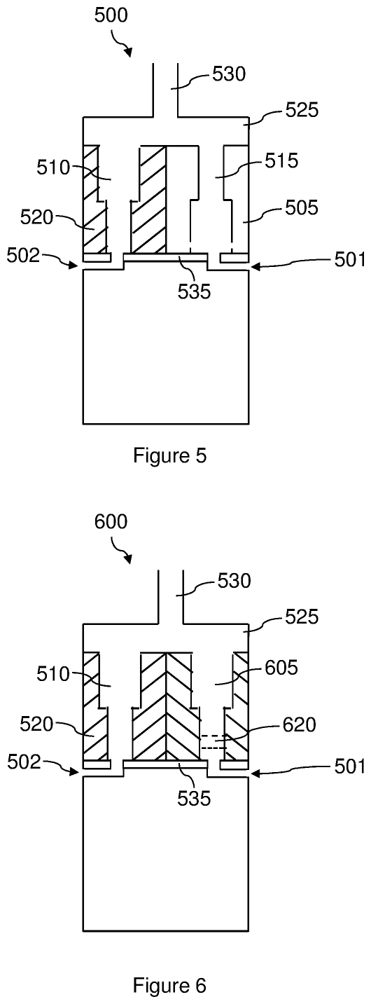

FIG. 5, which is not to scale, shows a schematic view of an embodiment of the device that is the subject of the present invention. This portable device 500 for vaporization of at least one active composition comprises:
at least one reservoir, 505 and/or 510, for storing an active composition;
for at least one reservoir, at least one vaporizer, 515 and/or 520, of the active composition contained in said reservoir;
at least one intermediate chamber 525 connected to at least one said reservoir, that can enable the vaporized active compositions to be mixed;
an inhalation line 530 connected to at least one intermediate chamber; and
an actuator 535 of at least one vaporizer according to a command received by the actuator.

Each reservoir, 505 and/or 510, is, for example, a sealed volume provided with an opening connected to the intermediate chamber 515. In some variants, each reservoir, 505 and/or 510, also comprises an inlet for supplying an active composition and/or an air inlet, 501 and/or 502. Each air inlet, 501 and/or 502, allows the passage of air when the patient sucks on the inhalation line 530.

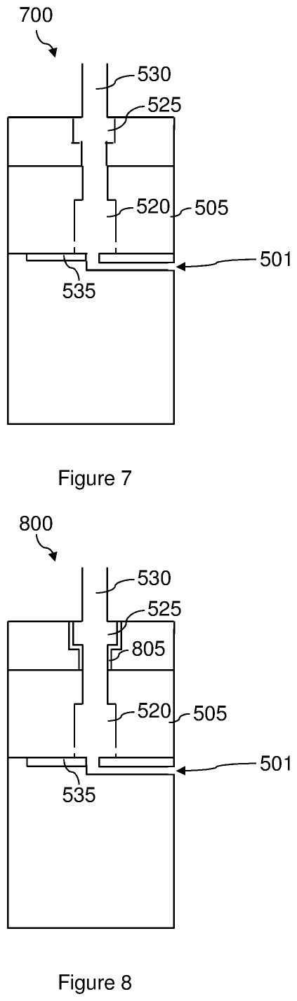

The device 500 shown in FIG. 5 comprises two reservoirs, 505 and 510. However, the device 500 can comprise a single reservoir, 505 or 510, or more than two reservoirs, at least one reservoir being connected to the intermediate chamber 525. An example of such a device with a single reservoir can be seen in FIGS. 7 and 8.

The intermediate chamber 525 is, for example, a sealed volume having at least two types of openings: one corresponding to openings connecting the intermediate chamber 525 to at least one reservoir, 505 and/or 510, and the other corresponding to the opening connecting the intermediate chamber 525 to the inhalation line 530.

For each reservoir, 505 and 510, the device 500 comprises a vaporizer, 515 and 520. Each vaporizer, 515 or 520, is configured to cause the evaporation of aromatic agents and the heated active composition contained in the reservoir, 505 or 510, associated with said vaporizer, 515 or 520.

In some embodiments, at least one vaporizer 520 is a cup vaporizer heated by conduction or convection.

When the cup is heated by conduction, a heating means raises the temperature of the walls of a reservoir that holds extracts of plants and/or dried plants, wax or an oil concentrate containing the active substance. These extracts of plants and/or dried plants, oil or wax are therefore heated by contact with the reservoir. When the user sucks on the device, the air passes over the extracts of plants and/or dried plants, oil or wax, which have been heated to a certain temperature, and carries the active substance(s) and/or aroma(s) into the respiratory tract of the user.

When the cup is heated by convection, a heating means located in front of the reservoir containing extracts of plants and/or dried plants, oil concentrate or wax is heated to a predefined temperature. Once this is reached the user is invited to inhale, which leads to the arrival of a flow of air that is instantly heated by the heating means before passing through the extracts of plants and/or dried plants, oil concentrate or wax in the reservoir. As the air is hot, this allows the active substance(s) and/or aroma(s) to be released so that the user can inhale them. This is referred to as convection because the extracts of plants and/or dried plants, oil or wax are heated by the heating means, but indirectly since it is the air that conducts the heat.

In some embodiments, at least one vaporizer 515 is a heating element vaporizer.

In some embodiments, at least one vaporizer is a heating mesh vaporizer, as shown in FIG. 6. The mesh is, for example, made of cotton, ceramic or metal. A gel comprising an active substance or essential oil is positioned on this mesh or impregnates this mesh.

When the device 500 comprises at least two reservoirs, 505 and 510, the device 500 can comprise any combination of vaporizers, 515 and 520, associated with each reservoir.

The association of two types of vaporizer enables the effects of these types of vaporizers to be combined during a single inhalation.

For example, a first reservoir is provided with a cup vaporizer or a heating mesh vaporizer and the content of the first reservoir consists, for example, of extracts of plants and/or dried plants and/or an active substance in a gel and/or an active substance concentrate and/or aromatic agents and/or essential oils. In some variants, the content of the first reservoir comprises dried tobacco leaves or cannabis and/or wax from plants, such as THC wax for example.

A second reservoir is provided with a heating element vaporizer and contains an aerosol base comprising, or not, an aromatic agent and/or an active substance. This aerosol base is, for example, water, PG (for “Propylene Glycol”) or VG (for “Vegetable Glycerin”), and/or an isotonic solution.

The advantage of a device as described with reference to FIG. 5 associating the heating of extracts of plants and/or dried plants, oil or wax and the vaporization of a diluent base is that this device makes it possible to associate the production of a vapor for recreational or therapeutic purposes and the vaporization of natural aromas and active compositions.

FIG. 6, which is not to scale, shows a schematic view of an embodiment of the device that is the subject of the present invention. This portable device 600 for vaporization of at least one active composition comprises:
at least one reservoir, 605 and/or 510, for storing an active composition;
for at least one reservoir, at least one vaporizer, 520 and/or 620, of the active composition contained in said reservoir;
at least one intermediate chamber 525 connected to at least one said reservoir, that can enable the vaporized active compositions to be mixed;
an inhalation line 530 connected to at least one intermediate chamber; and
an actuator 535 of at least one vaporizer according to a command received by the actuator.

This FIG. 6 is a variant of FIG. 5 in which one of the vaporizers 620 is a heating mesh vaporizer.

The advantage of associating two systems that each heat an extract of plants and/or dried plants, oil or wax is that this offers greater flexibility in the use of the device. For example, this device allows an optimum heating temperature to be selected for each of the elements.

FIG. 7, which is not to scale, shows a schematic view of an embodiment of the device that is the subject of the present invention. This portable device 700 for vaporization of at least one active composition comprises:
at least one reservoir 505 for storing an active composition;
for at least one reservoir, at least one vaporizer 520 of the active composition contained in said reservoir;
at least one intermediate chamber 525 connected to at least one said reservoir, that can enable the vaporized active compositions to be mixed;
an inhalation line 530 connected to at least one intermediate chamber; and
an actuator 535 of at least one vaporizer according to a command received by the actuator.

In this embodiment, the intermediate chamber 525 stores a heated compound following the activation of at least one vaporizer 520 for active composition contained in a reservoir 505. The heating can result from putting the compound in contact with the hot vapors and/or by the compound's proximity to a heating element of the vaporizer 520.

FIG. 8, which is not to scale, shows a schematic view of an embodiment of the device that is the subject of the present invention. This portable device 800 for vaporization of at least one active composition comprises:
at least one reservoir 505 for storing an active composition;
for at least one reservoir, at least one vaporizer 520 of the active composition contained in said reservoir;
at least one intermediate chamber 525 connected to at least one said reservoir, that can enable the vaporized active compositions to be mixed;
an inhalation line 530 connected to at least one intermediate chamber; and
an actuator 535 of at least one vaporizer according to a command received by the actuator.

In this embodiment, the intermediate chamber 525 comprises an element 805 heating the compound stored in said chamber. This heating element 805 is, for example, an electrical resistor. In the embodiment shown in FIG. 8, the heating element 805 is positioned near a mesh or a cup containing the compound. In some variants, the heating element 805 is positioned at least in part around and/or in the center of the compound.

The intermediate chamber 525 as described with reference to FIGS. 7 and 8 can preferably be removed from the device 700 and 800.

The devices described with reference to FIGS. 7 and 8 make it possible to associate the production of a vapor, for recreational or therapeutic purposes, and the delivery of active and aromatic substances. The vapor, in passing through the material to be heated, will itself be infused with the aromas and active substances whose release it enables.

The device described with reference to FIG. 8 makes it possible to obtain a warmer vapor and to extract the active substance more easily than in the case of FIG. 7.

Therefore, as can be understood, the device that is the subject of the present invention can comprise at least two separate aerosolization means. These two aerosolization means can each be associated with a separate reservoir, the device therefore comprising at least two reservoirs. The two aerosolization means can be of the same type or of different types.

In some variants, such as the one shown in FIG. 1, at least a first aerosolization means 130 is a nebulizer and at least a second aerosolization means 135 is a nebulizer.

In some variants, such as the one shown in FIG. 4, at least a first aerosolization means 415 is a means for expanding a pressurized content and at least a second aerosolization means 420 is a means for expanding a pressurized content. Such an expansion means corresponds, for example, to an opening in a reservoir commanded by a regulator, as described with reference to FIG. 4.

In some variants, at least two aerosolization means utilize a different aerosolization technique.

In some variants, such as the one shown in FIG. 2, at least a first aerosolization means 230 is a nebulizer and at least a second aerosolization means 235 is a vaporizer.

In some variants, such as the one shown in FIG. 3, at least a first aerosolization means 130 is a nebulizer and at least a second aerosolization means 306 is a means for expanding a pressurized content in a reservoir.

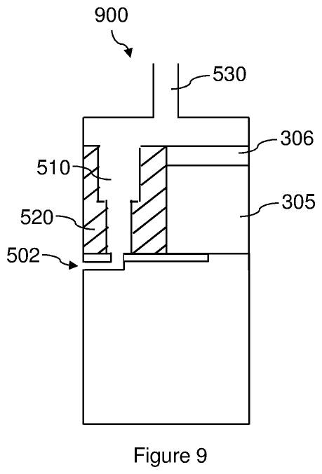

In some variants, such as the one shown in FIG. 9, at least a first aerosolization means 520 is a vaporizer and at least a second aerosolization means 306 is a means for expanding a pressurized content in a reservoir. Such a vaporizer 520 is described, for example, with reference to FIG. 5, while such an expansion means 306 is described with reference to FIG. 3. Such an expansion means corresponds, for example, to an opening in a reservoir commanded by a regulator, as described with reference to FIG. 3.

1-29. (canceled)

30. Portable device for inhalation of at least one active composition, comprising:
at least one reservoir for storing an active composition;
at least one intermediate chamber connected to at least one reservoir;
an inhalation line connected to the intermediate chamber;
a means for aerosolizing the active composition coming from a reservoir; and
a regulator) for regulating the quantity of aerosolized active composition according to a command representing a quantity of active composition to be aerosolized received by the regulator.

31. Device according to claim 30, wherein at least one aerosolization means is a nebulizer for liquid active composition stored in a reservoir.

32. Device according to claim 31, wherein a nebulizer comprises a screen having openings, and a means for making the screen oscillate, the regulator comprising a means for closing at least one portion of the openings according to the quantity of active composition to be aerosolized.

33. Device according to claim 31, wherein the nebulizer comprises a means for making a surface oscillate, the regulator being configured to make the surface oscillate at a frequency determined as a function of the quantity of active composition to be aerosolized and/or of characteristics of the active composition to be nebulized.

34. Device according to claim 33, wherein the surface is a screen having openings, the regulator being configured to make the screen oscillate at a frequency determined as a function of the quantity and/or characteristics of the active composition to be nebulized or wherein the surface is located in the bottom portion of the reservoir so as to transmit vibrations to the active composition, the regulator being configured to command the oscillation at a frequency determined as a function of the quantity and/or characteristics of the active composition to be nebulized or wherein the nebulizer comprises a plurality of screens having different porosities, and a means for making each screen oscillate independently, the regulator being configured to command the oscillation of at least one of the screens as a function of the quantity and/or characteristics of the active composition to be aerosolized.

35. Device according to claim 30, which comprises a vaporizer for the active composition stored in the reservoir.

36. Device according to claim 30, which comprises at least two reservoirs holding an active composition to be aerosolized, one holding an active composition containing an active substance and the other holding an active composition containing a lower, or zero, concentration of said active substance, each aerosolization means associated with a said reservoir being controlled as a function of the quantity and/or characteristics of the active composition to be aerosolized.

37. Device according to claim 31, which comprises a pressurized reservoir, holding oxygen or any other pressurized gas that can contain an active substance in the form of a solution or suspension, connected to the intermediate chamber, the aerosolization of the oxygen or pressurized gas being controlled by the regulator.

38. Device according to claim 30, wherein at least two reservoirs are pressurized, one holding a pressurized active composition containing an active substance and the other holding a pressurized active composition containing a lower, or zero, concentration of said active substance, at least one reservoir being connected to the intermediate chamber acting as expansion chamber for the fluids being released there.

39. Device according to claim 38, wherein the regulator is configured to command a duration for aerosolizing the active composition contained in at least one of the two reservoirs as a function of a quantity of active substance, contained in the composition, to be delivered.

40. Device according to claim 30, which comprises at least two aerosolization means, are each connected to a separate reservoir, the device comprising at least two reservoirs, each reservoir containing a different active composition.

41. Device according to claim 40, wherein at least the first and second aerosolization means are each nebulizers or each means for expanding a pressurized content.

42. Device according to claim 40, wherein at least two aerosolization means utilize a different aerosolization technique.

43. Device according to claim 42, wherein at least a first aerosolization means is a nebulizer and at least a second aerosolization means is a vaporizer.

44. Device according to claim 43, wherein at least a first aerosolization means is a nebulizer and at least a second aerosolization means is a means for expanding a pressurized content in a reservoir.

45. Device according to claim 43, wherein at least a first aerosolization means is a vaporizer and at least a second aerosolization means is a means for expanding a pressurized content in a reservoir.

46. Device according to claim 35, wherein at least one vaporizer is a cup vaporizer heated by conduction or convection and/or at least one vaporizer is a heating mesh vaporizer.

47. Device according to claim 35, wherein the intermediate chamber stores a heated compound following the activation of at least one vaporizer for active composition contained in a reservoir.

48. Device according to claim 47, wherein the intermediate chamber comprises an element heating the compound stored in said chamber.

49. Device according to claim 47, wherein the heating element is positioned near a mesh or a cup containing the compound or positioned at least in part around and/or in the center of the compound.
